(19) United States
(12) Patent Application Publication
(10) Pub. No.: US 2025/0160987 A1
(43) Pub. Date: May 22, 2025

(54) SYNCHRONIZED MOTION OF INDEPENDENT SURGICAL DEVICES

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Charles J. Scheib, Loveland, OH (US); Kevin M. Fiebig, Cincinnati, OH (US); Taylor W. Aronhalt, Loveland, OH (US)

(21) Appl. No.: 18/809,890

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/00; A61B 1/00; A61B 1/313; A61B 17/29; A61B 34/30; A61B 34/32

(52) U.S. Cl. CPC A61B 34/77; A61B 1/00149; A61B 17/29; A61B 34/32; A61B 1/3132; A61B 2034/303

(57) ABSTRACT

Devices, systems, and techniques for synchronized motion of independent surgical devices. An example first surgical instrument may have a movable component. The first surgical instrument may receive a motion control parameter. The motion control parameter may be one or more of: a force relative to a tissue contact shared between the first surgical instrument and the second surgical instrument; or a positional relationship between the first surgical instrument and the second surgical instrument. The first surgical instrument may sense a motion event caused by a user control input to a second surgical instrument. The first surgical instrument may autonomously adjust motion of the movable component based on the motion control parameter and the motion event.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/810,133, filed Aug. 20, 2024, and
U.S. patent application Ser. No. 18/810,170, filed Aug. 20, 2024.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst case scenario, injure a patient.

Feature(s) described herein relate to techniques for synchronized motion between surgical devices to manage the interaction between them. For example, multiple devices (e.g., which may have different manufacturers and/independent control systems) may actively synchronize their motions. A user may have simultaneous hybrid control of multiple separate instruments controlled by two independent smart systems. The user may, for example, control the instruments from a single control station.

A device may determine its movements based on movement of another device. For example, a first device may be actively controlled by the user and a second device may be put into a “follow-me” mode in which the second device maintains a certain proximity to the moving first device. The interdependent motions may have limits that are derived from each other. For example, two devices may be configured to maintain a tissue tension. In this case, if a user manually increases the force a first device is applying to the tissue, a second device that is also in contact with the tissue may autonomously reduce the force applied by the second device so that the overall tissue tension remains relatively stable. Similarly, hybrid load-stroke and/or a proportionate, integral, derivative (PID) control loop may be used to maintain the relationship between devices.

In another example, a first device may be put into a “station-keeping” or “position-holding” mode in which the first device maintains its absolute location in a global reference plane. A user may therefore know where the first device is at all times because the location is constant. This may allow the user to move a second device in the vicinity of the first device without causing an unwanted interaction between the devices.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates an example surgical operating room with robotic surgical instruments.

FIG. 6A illustrates example smart surgical devices

FIG. 6B illustrates example legacy devices.

FIG. 7 is a block diagram illustrating example components of a surgical device.

FIG. 8A is a block diagram illustrating an example control loop in a surgical device.

FIG. 8B illustrates an example of a surgical instrument autonomously moving to maintain a proximity boundary with another surgical instrument.

FIG. 8C illustrates an example of a scope autonomously moving to maintain another surgical instrument in a field of view of the scope.

FIGS. 9A and 9B illustrate an example tumor removal procedure using techniques described herein.

FIGS. 10A-C illustrate another example tumor removal procedure using techniques described herein.

FIGS. 11A-C illustrate yet another example tumor removal procedure using techniques described herein.

FIG. 12 illustrates an example method that may be performed by a surgical instrument.

### DETAILED DESCRIPTION

A more detailed understanding may be had from the following description, given by way of example in conjunction with the accompanying drawings.

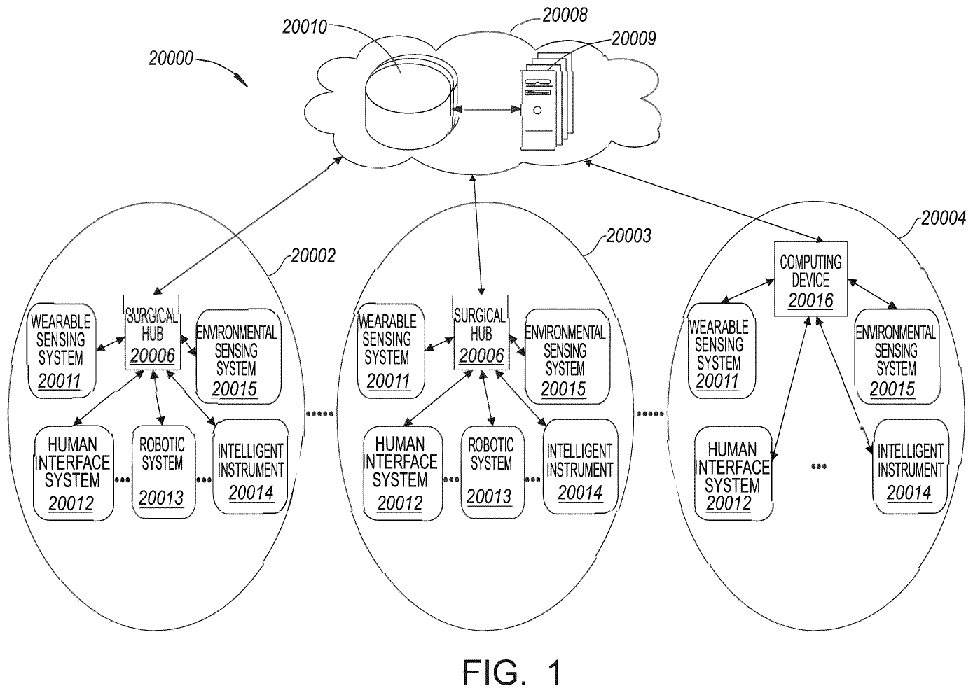

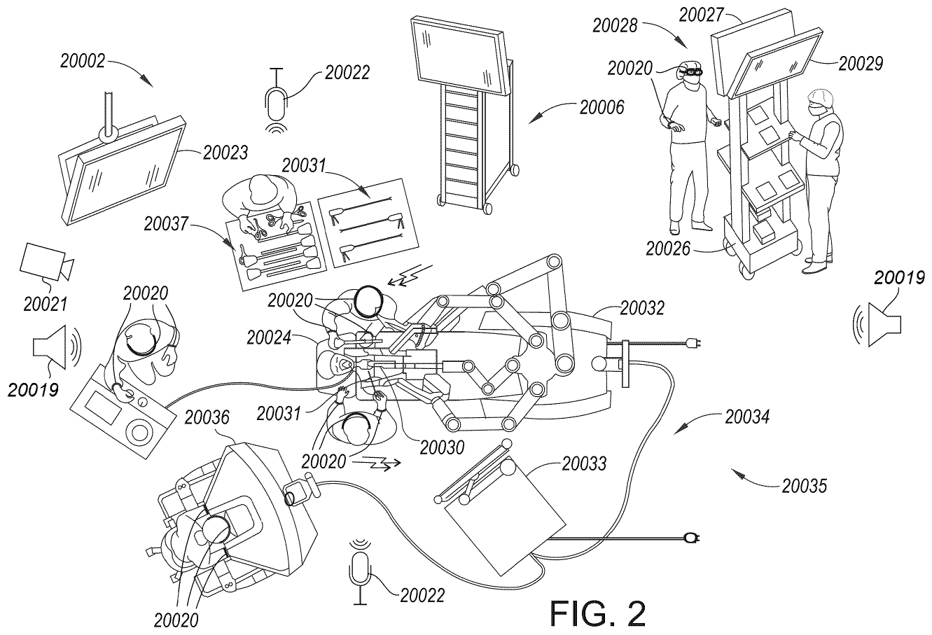

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiogramcephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

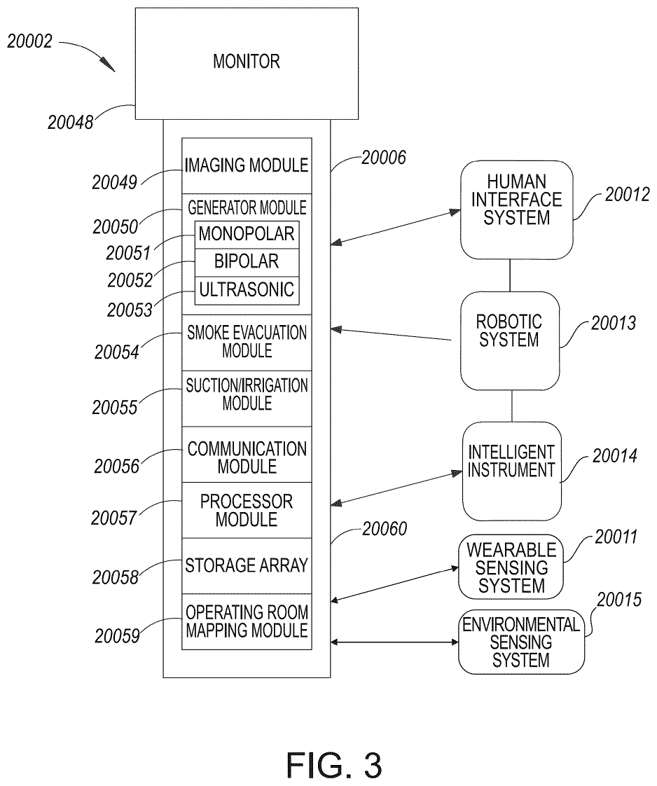

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

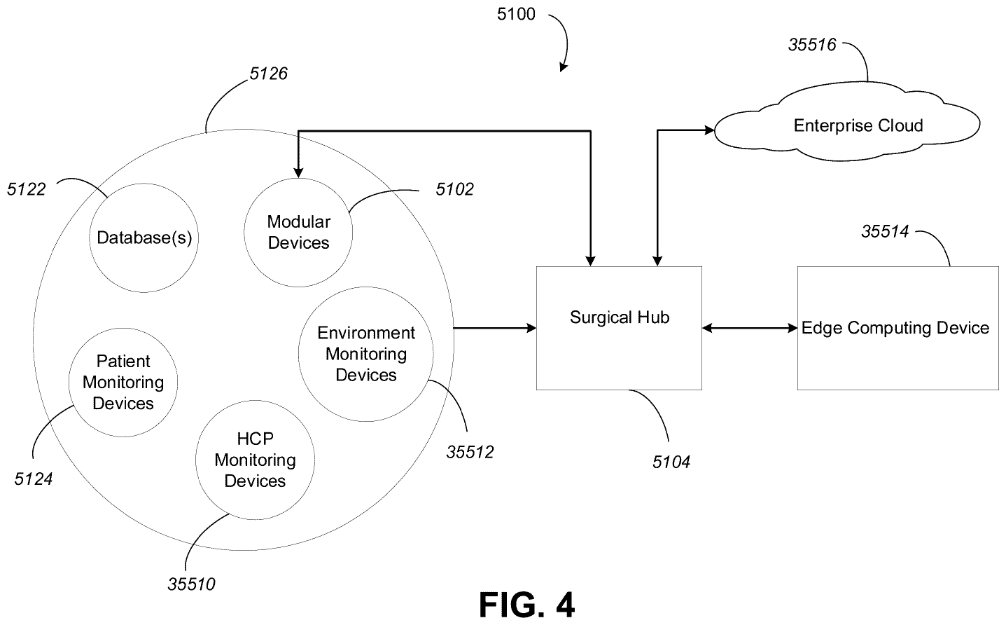

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiogramonitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

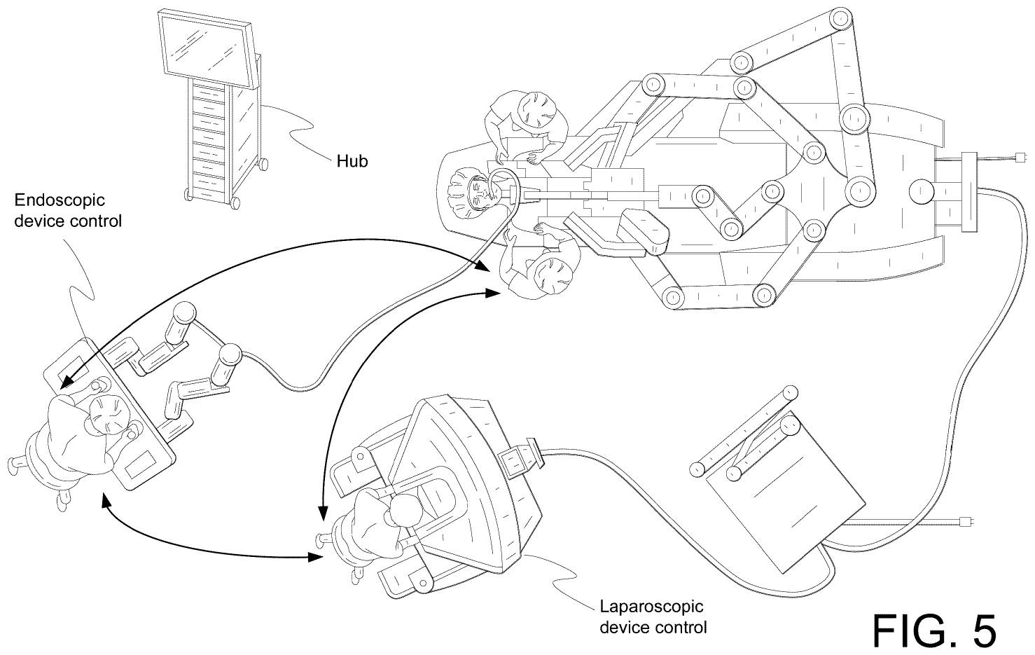

FIG. 5 illustrates an example surgical operating room with robotic surgical instruments. As shown, a user (e.g., a surgeon) may move from one device to another during an operation. In the example of FIG. 5, the surgeon is shown moving between a laparoscopic device control panel, an endoscopic device control panel, and a manual device (e.g., legacy device located in proximity to the patient).

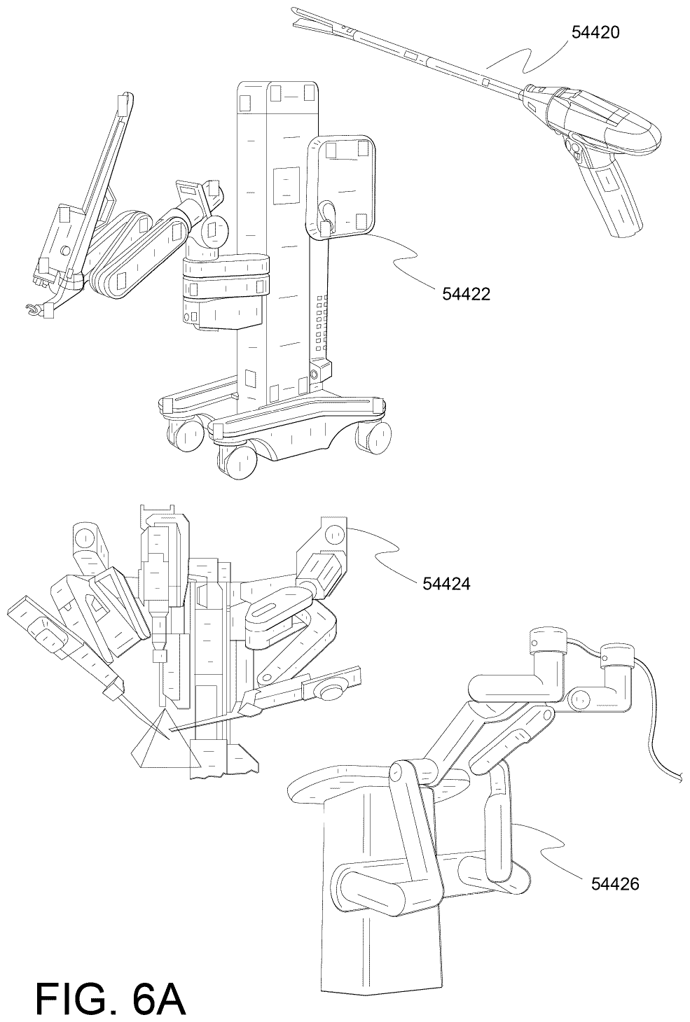

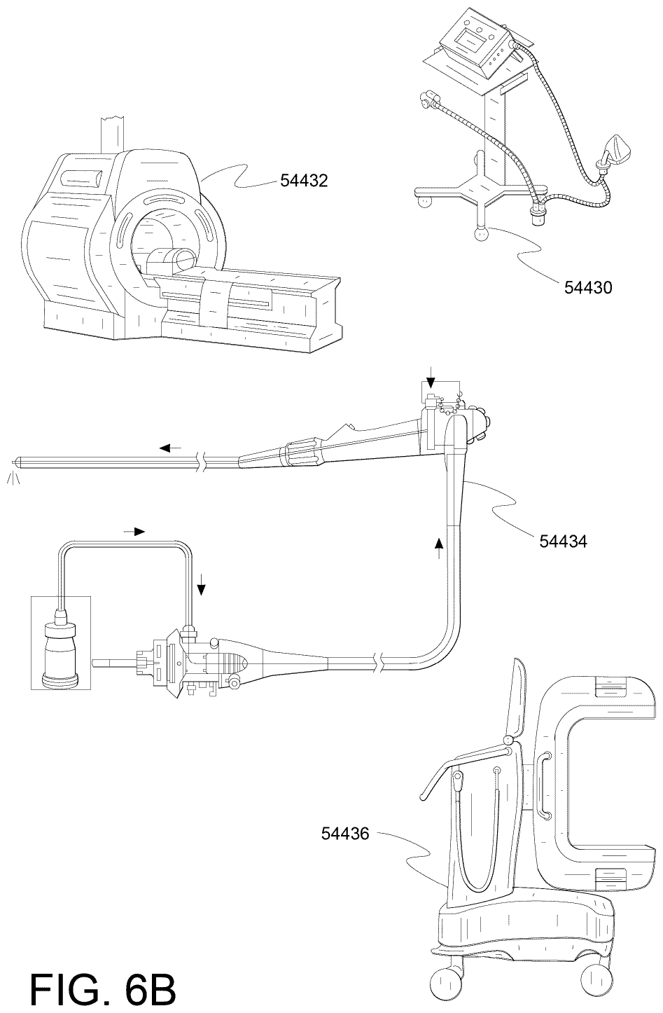

FIG. 6A illustrates example smart surgical devices. For example, smart devices may include a smart surgical stapler 54420, a smart robotic arm (e.g., Hugo robot) 54422, a multi-arm robot (e.g., da Vinci robot) 54424, and/or a smart flexible endoscope (e.g., Monarch flexible endoscope) 54426.

FIG. 6B illustrates example legacy surgical devices. For example, legacy devices may include a ventilator 54430, a magnetic resonance imaging (MRI) machine 54432, a manual endoscope 54434, and/or a computed tomography (CT) machine (e.g., cone-beam CT scanner) 54436.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst case scenario, injure a patient.

A system (e.g., a dual system) may have independent yet simultaneous control of more than one (e.g., two) smart instruments (e.g., by the same user). Feature(s) described herein may provide medical professionals the ability to operate an actuatable instrument from a first robot (e.g., endoscopic working channel tool) at the same time as a second actuatable instrument from a second robot (e.g., a lap powered device). For example, operating multiple actuatable devices at once (e.g., an endoscopic device and a laparoscopic device) may be used to hand off a piece of tissue or anatomy from one to the other.

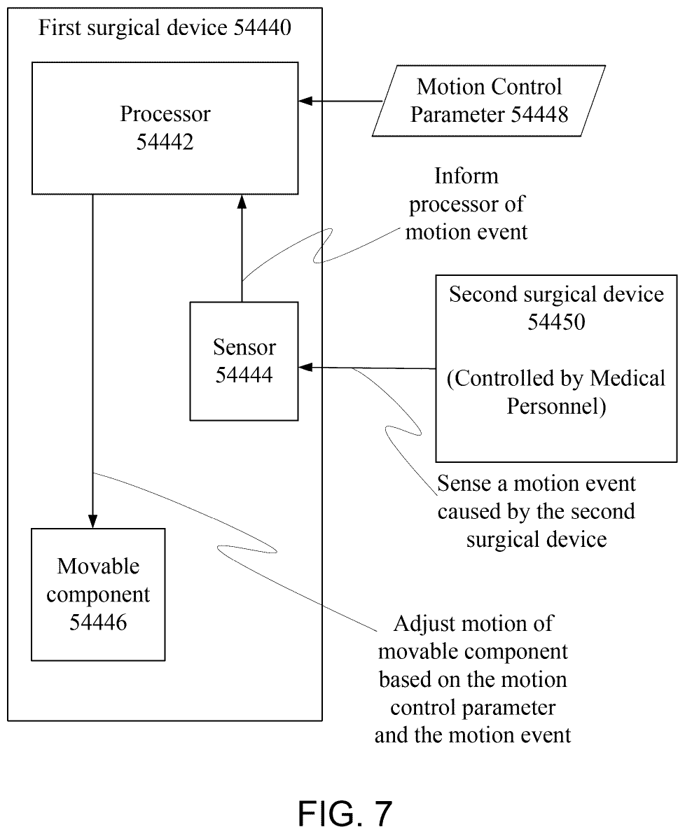

As illustrated in FIG. 7, a first surgical device 54440 may include a processor 54442, a sensor 54444, and at least one movable component 54446. The first surgical device 54440 may be (semi) autonomously controlled. The first surgical device 54440 may be in proximity to a second surgical device 54450, which may be controlled by medical personnel (e.g., a surgeon). The first surgical device 54440 may receive a motion control parameter 54448. For example, the motion control parameter 54448 may be preconfigured or determined based on a user input. The motion control parameter 54448 may be a minimum and/or maximum distance to be maintained between the first and second surgical devices, an indication for the first surgical device 54440 to keep the second surgical device 54450 in its field of view, and indication to maintain a constant tissue tension between the first and second surgical devices, and/or the like.

As illustrated in FIG. 7, the sensor 54444 may sense a motion event caused by the second surgical device (e.g., movement of the second surgical device, a shift in tissue tension, etc.). The sensor 54444 may inform the processor 54442 of the detected motion event. The processor 54442 may analyze the motion event in view of the motion control parameter 54448. The processor 54442 may autonomously adjust motion of the movable component 54446 based on the motion control parameter 54448 and the motion event. For example, the processor 54442 may autonomously adjust motion of the movable component by determining an effect of the motion event based on a quantity defined by the motion control parameter; and determining a way in which to adjust the motion of the moveable component to provide a corresponding canceling effect to cancel out the determine effect of the motion event.

For example, if the motion event indicates that the second surgical device 54450 is moving away from the first surgical device 54440 and the motion control parameter 54448 indicates a maximum distance between the surgical devices, the processor 54442 may move the movable component 54446 toward the second surgical device 54450 to keep the devices within the maximum distance from each other. Similarly, if the motion event indicates a decrease in tissue tension for a piece of tissue being held by the first and second surgical devices and the motion control parameter indicates a fixed tissue tension, the movable component 54446 may move away from the second surgical device 54450 to increase the tissue tension back to the original tissue tension. In examples, the first surgical instrument may be an endoscopic instrument and the second surgical instrument may be a laparoscopic instrument.

In an example, the movable component may be a grasping device, the motion event may be a change in tissue tension associated with tissue held by the grasping device, and the motion control parameter may be a tensile range. In this example, adjusting motion of the movable component based on the motion control parameter and the motion event may involve adjusting a load control of the movable component to keep the tissue tension within the tensile range.

In another example, the motion event may be the second surgical instrument moving away from the first surgical instrument, and the motion control parameter may be a maximum distance between the first surgical instrument and the second surgical instrument. In this example, adjusting motion of the movable component based on the motion control parameter and the motion event may involve moving the movable component toward the second surgical instrument to keep a distance between the first surgical instrument and the second surgical instrument below the maximum distance.

In yet another example, the motion event may be the second surgical instrument moving towards the first surgical instrument, and the motion control parameter may be a minimum distance between the first surgical instrument and the second surgical instrument. In this example, adjusting motion of the movable component based on the motion control parameter and the motion event may involve moving the movable component away from the second surgical instrument to keep a distance between the first surgical instrument and the second surgical instrument above the minimum distance.

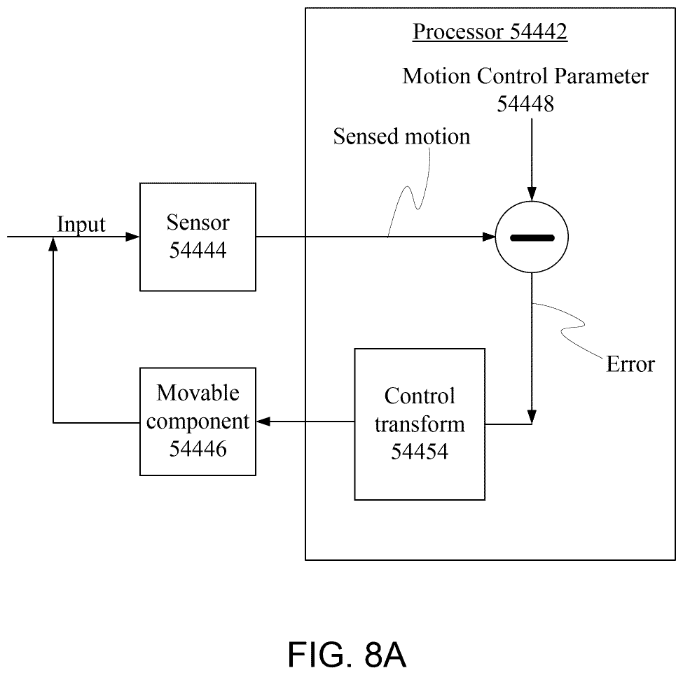

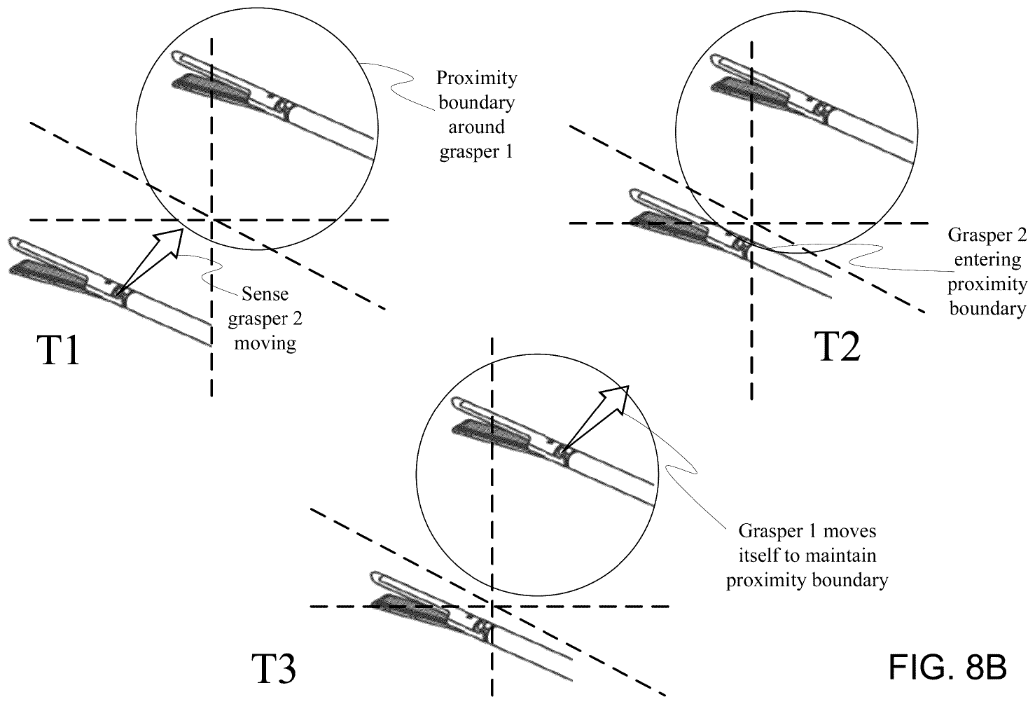

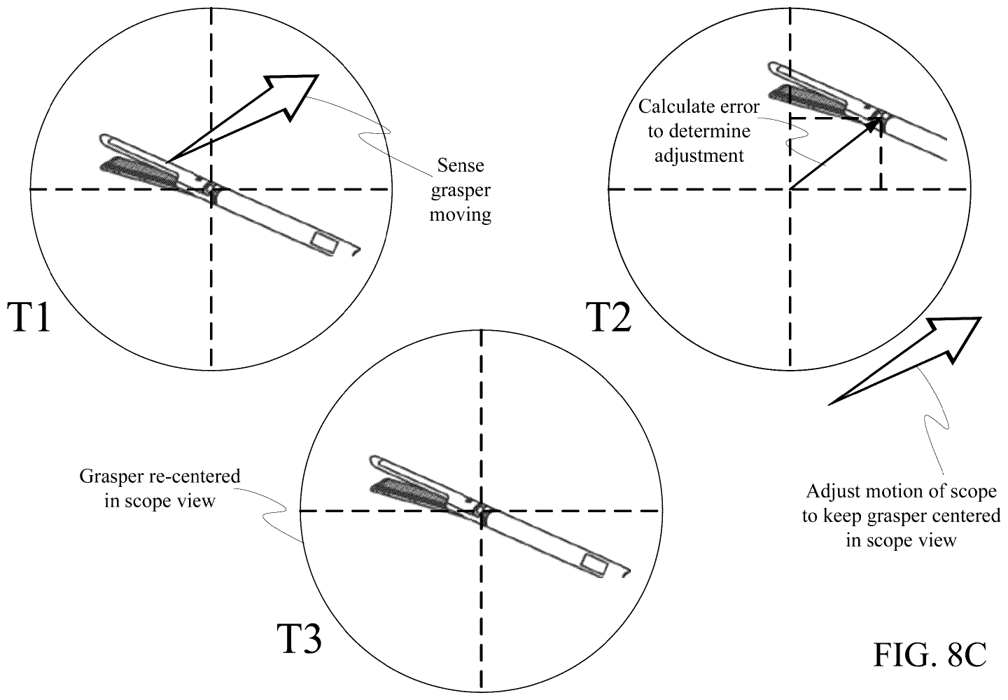

FIG. 8A is a block diagram illustrating an example control loop in a surgical device (e.g., the first surgical device 54440 of FIG. 7). As illustrated in FIG. 8A, the device may receive an input (e.g., an external input) that may be sensed by a sensor 54444. The sensor 54444 may determine, based on the input, a sensed motion event. The sensor 54444 may indicate the sensed motion to the processor 54442. The processor 54442 may compare the sensed motion to the motion control parameter 54448 to determine an error (e.g., a differential error between the motion control parameter 54448 and an effect caused by the sensed motion). The processor may use the error as an input to a control transform 54454. The control transform 54454 may output a motion for the movable component 54446 to perform. The processor 54442 may indicate for the movable component 54446 to perform the motion. The motion of the second surgical instrument may continue or may stop while the movable component 54446 is moving. The sensor may continue to monitor the motion of the second surgical instrument (e.g., relative to the movable component 54446, which may be moving as well). The control loop may repeat until the error is within an acceptable range.

FIG. 8B illustrates an example of a surgical instrument autonomously moving to maintain a proximity boundary with another surgical instrument. A first grasper (e.g., grasper 1) may be in proximity to a second grasper (e.g., grasper 2). Grasper 1 may have a proximity boundary around it (e.g., the control parameter may indicate for grasper 1 to maintain at least a minimum distance away from other devices). At a time T1, grasper 1 may sense that grasper 2 is moving toward grasper 1. At a time T2, grasper 1 may determine that grasper 2 is about to enter the proximity boundary around grasper 1. In response, at a time T3, grasper 1 may autonomously move away from grasper 2 to maintain the minimum distance between the graspers.

In an example, the moveable component may be a scope, the motion event may be the second surgical instrument is moving out of a field of view of the scope, and the motion control parameter may be a maximum distance that the second surgical instrument can be away from the center of the field of view of the scope. In this example, adjusting motion of the movable component based on the motion control parameter and the motion event may involve moving the scope to keep the second surgical instrument within the maximum distance away from the center of the field of view of the scope.

FIG. 8C illustrates an example of a scope autonomously moving to maintain another surgical instrument in a field of view of the scope. As shown, the scope may have a grasper centered in the scope's field of view. The scope may be used to allow a surgeon to monitor where the grasper is relative to a target structure (e.g., a tumor). At a time T1, the scope may sense that the grasper is moving. At a time T2, after the grasper has moved, the scope may calculate the error between the center of the field of view and the location of the grasper in the field of view. The scope may adjust its motion, based on the error, to keep the grasper centered in the scope field of view. At a time T3, the scope may have moved to re-center the grasper in the scope field of view.

The robot console control may have a hybrid control with a first controller operating a first robot arm (e.g., coupled to a first robot) and a second controller operating a second robot arm (e.g., coupled to a second robot). The system may include a display. The display may be a side-by-side display (e.g., from each of the independent robot visualization means). The display may be a composite display (e.g., where one of the visualization streams is augmented with the other display to form one composite display). The composite display may have the ability to shift to a percent (e.g., any percent) of the two hybridized views (e.g., so the user may customize the angle and/or level of transparency to see both approaches and instruments simultaneously on a single imaging means).

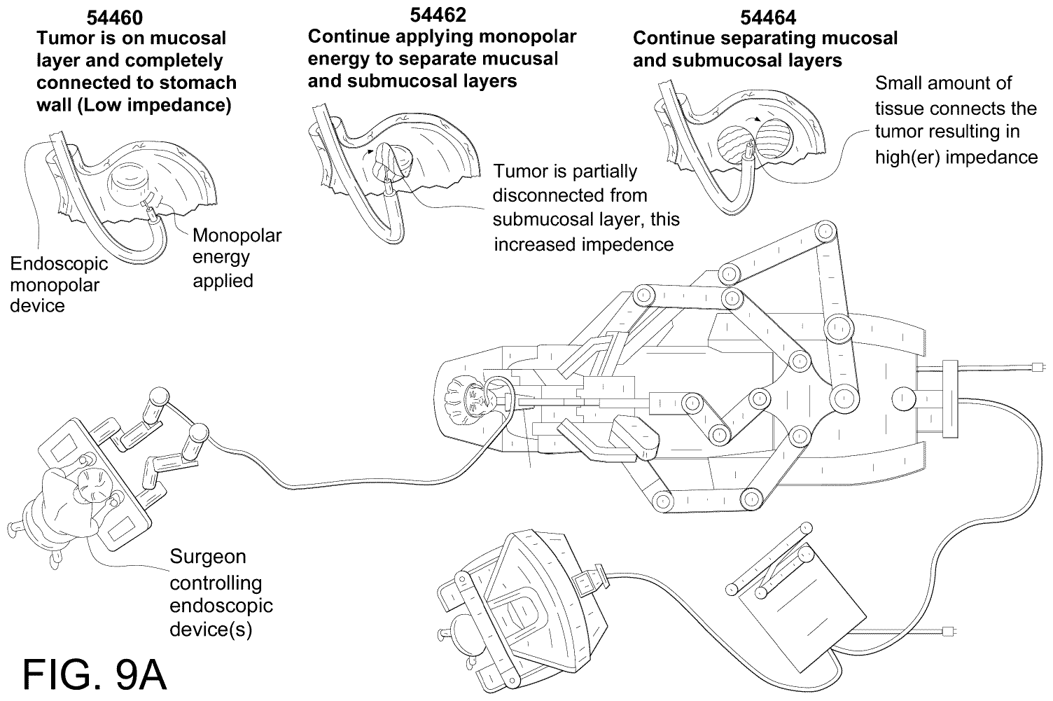

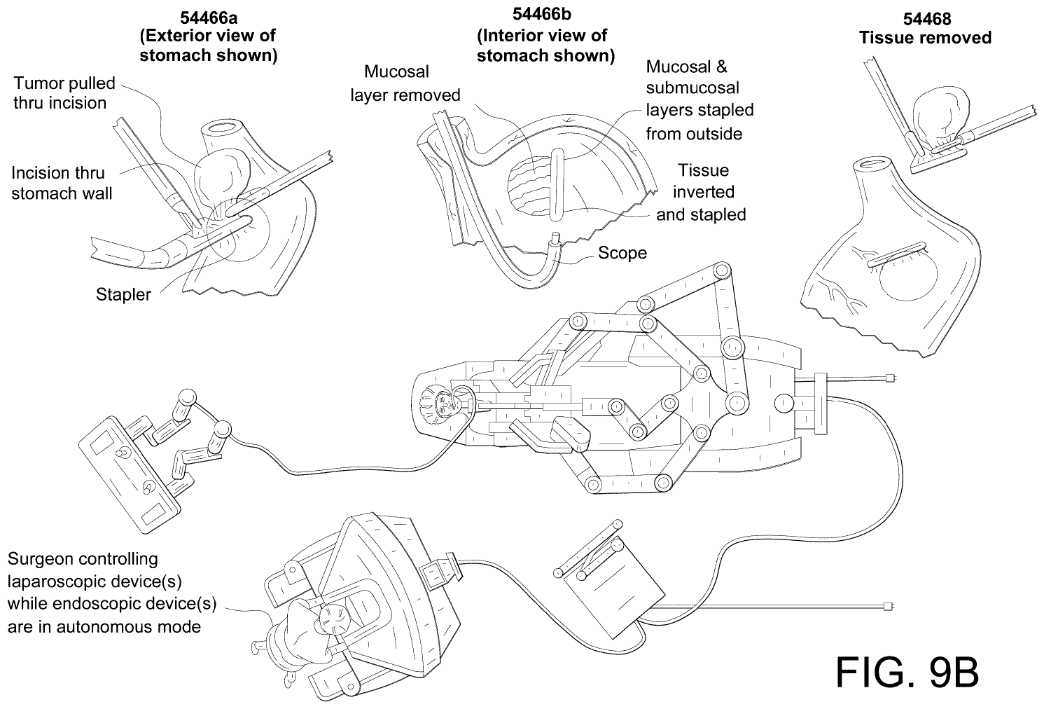

For example, a flexible endoscopic scope (e.g., endoscope) may be used to resect (e.g., mucosally resect) a tumor within a patient's stomach from the serosal layer (e.g., with a monopolar blade). FIGS. 9A and 9B illustrate this procedure. At 54460-54464, the surgeon may be controlling endoscopic device(s) (e.g., smart endoscope and/or monopolar energy device) from a first control station. At 54460, the tumor may be completely connected to the stomach wall in the mucosal layer. At this time, the monopolar energy device may experience relatively low impedance as it begins applying the monopolar energy to the stomach lining.

At 54462, the monopolar energy device may continue to apply the monopolar energy to separate the mucosal and submucosal layers. As the tumor is separated from the submucosal layer, the monopolar energy device may experience increased impedance. At 54464, the monopolar energy device may continue to apply the monopolar energy to separate the mucosal and submucosal layers. At this point, the monopolar energy device may be experiencing a relatively high impedance.

Once tumor is separated about ¾ of the way, the monopolar device may be removed from the working channel. The tumor may then be removed laparoscopically, as illustrated in FIG. 9B. As shown, the surgeon may control the laparoscopic device(s) at a second control station (e.g., a control station completely separate from the first control station used for the endoscopic devices). To control the tumor and perform the flip to the laparoscopic side, one or more grasper(s) may be introduced. The grasper(s) may be used to grasp the stomach near the internal grasping point. The grasper(s) may be used to cut the opening. The grasper(s) may be used to grasp the tumor adjacent to the remaining connection to the stomach. The grasper(s) may then be placed in a station keeping mode.

Once the opening is created, the surgeon may hybridize the approach. For example, the surgeon may release the grasper holding the overall stomach into a station keeping mode. The surgeon may replace control of the grasper (e.g., laparoscopic grasper) with a console control associated with a flexible endoscopic grasper. The endoscopic grasper may therefore be moved at the same time as the remaining laparoscopic grasper. The remaining laparoscopic grasper may be inserted into the incision. The endoscopic grasper may hand off the tumor to the laparoscopic grasper (not shown in FIG. 9B). The laparoscopic grasper may be retracted through (e.g., back through) the incision. The endoscopic grasper may push to get the tumor through the incision (e.g., following the laparoscopic grasper). At 54466a-b, the grasper(s) may hold the tumor while a surgical stapler staples the incision closed. The tumor may then be completely separated from the stomach and removed using the laparoscopic grasper(s), as shown at 54468.

The displays of the two systems (e.g., the endoscopic and laparoscopic systems) may be shown as a side-by-side (e.g., with an integrated overlap and sizing). The integrated overlap and size may allow the portions (e.g., two portions) of the image to join as a synchronized hybrid image. The synchronized hybrid image may prevent a mismatch from confusing the size (e.g., rapid changes in scale due to differing magnifications or alignments) of an object (e.g., in this example, the tumor) as it is passed from one side to the other (e.g., internal to external, for example, through the incision).

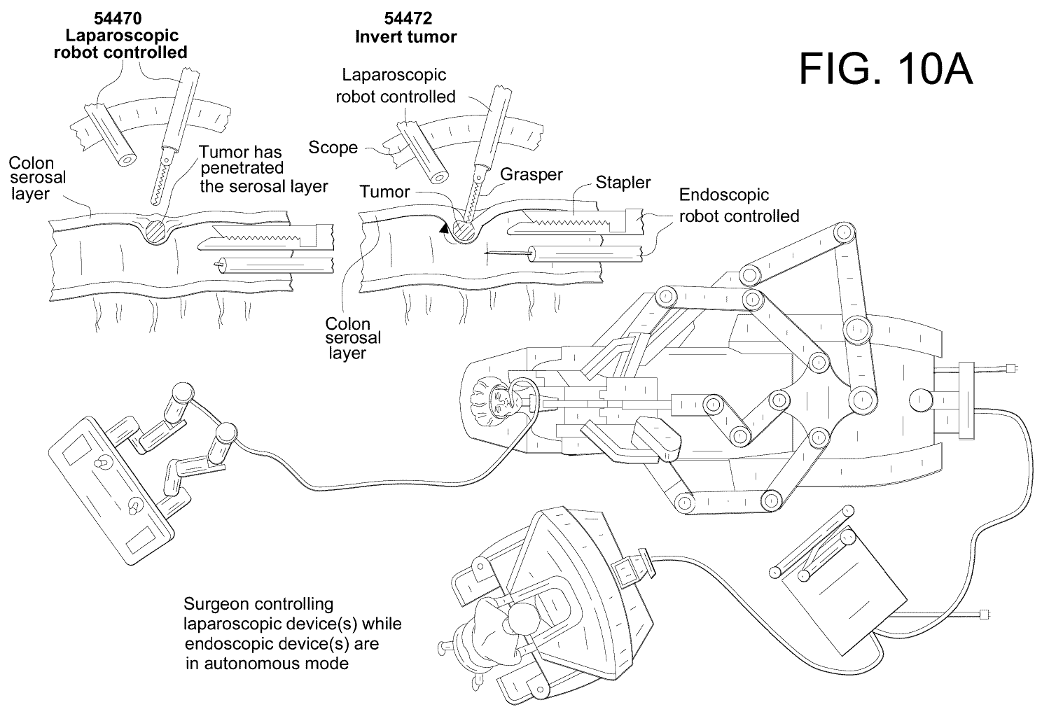

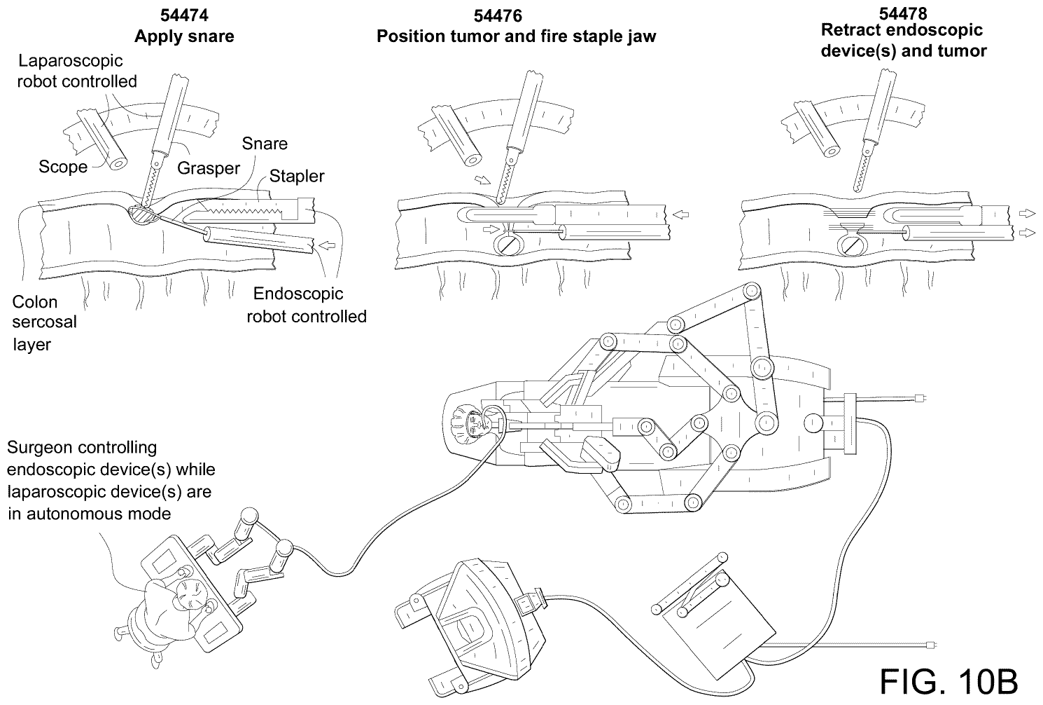

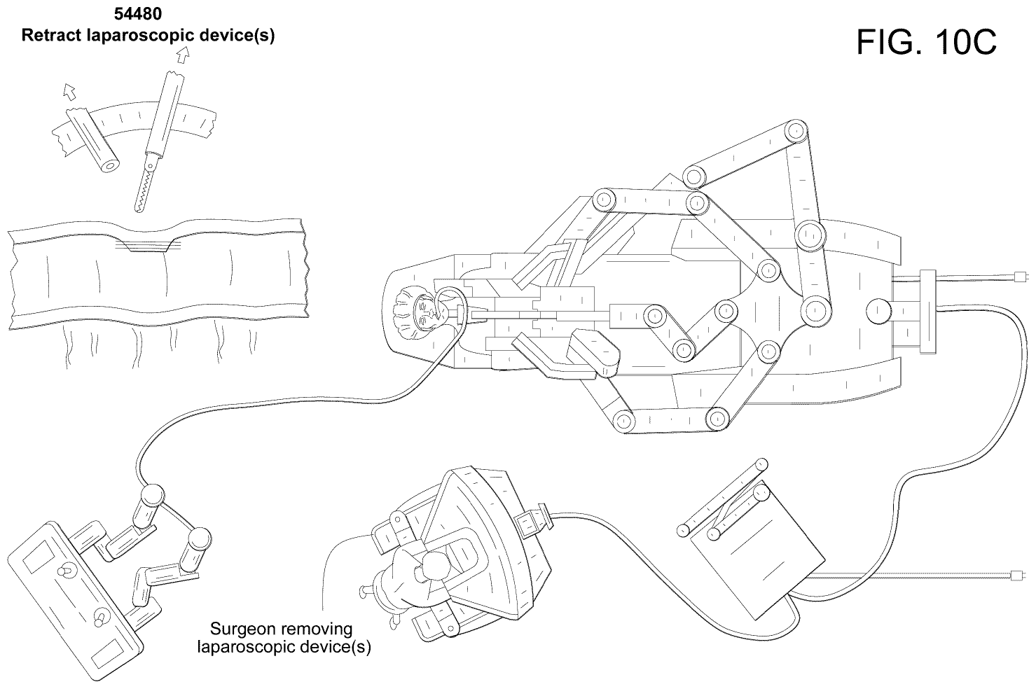

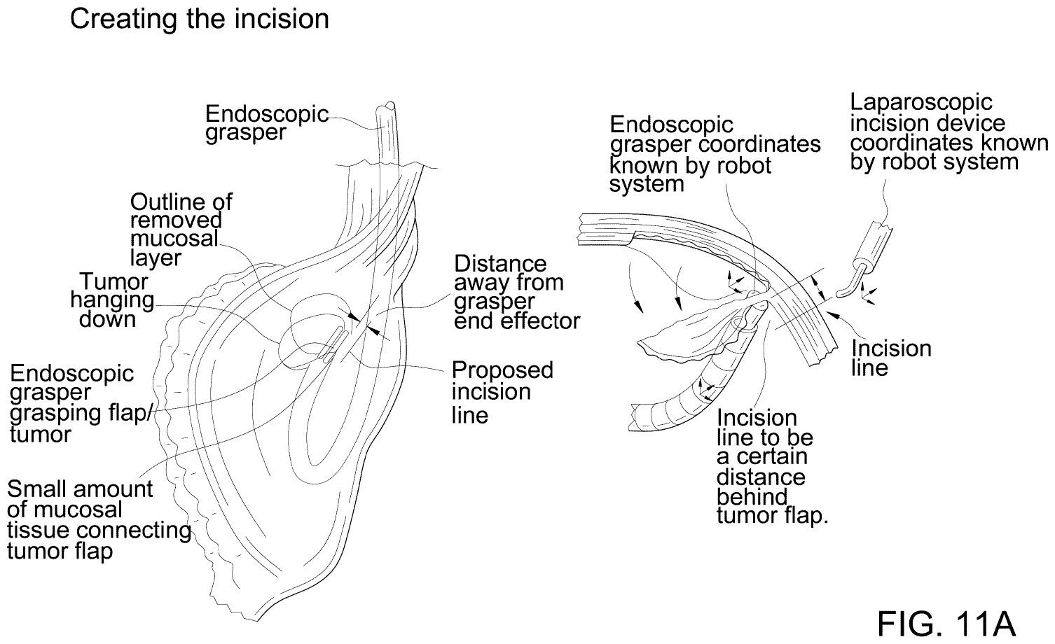

FIGS. 10A-C illustrate another example hybrid surgical procedure (e.g., a colorectal tumor removal). As shown at 54470, both endoscopic and laparoscopic device(s) may be inserted into a patient. During the actions illustrated in FIG. 10A, a surgeon may control the laparoscopic device(s), while the endoscopic device(s) are held in an autonomous (e.g., station-keeping) mode. At 54472, the surgeon may use a laparoscopic grasper to push the tumor further into the colon (e.g., toward the endoscopic device(s)).

As shown in FIG. 10B, the surgeon may move to a second control station (e.g., separate from the first control station) to control the endoscopic device(s). The laparoscopic device(s) may be placed in an autonomous (e.g., station-keeping) mode. At 54474, the surgeon may apply an endoscopic snare to the inverted tumor. At 54476, the surgeon may retract the snare to pull the tumor further into the colon. The snare may be clenched to reduce the amount of colon tissue that is removed. At 54476, the surgeon may fire an endoscopic stapler to separate the tumor from the colon tissue and staple the resulting incision. At 54478, the surgeon may retract the endoscopic device(s) and the tumor. As illustrated in FIG. 10C, the surgeon may move back to the first control station (controlling the laparoscopic devices). At 54480, the surgeon may retract the laparoscopic device(s).

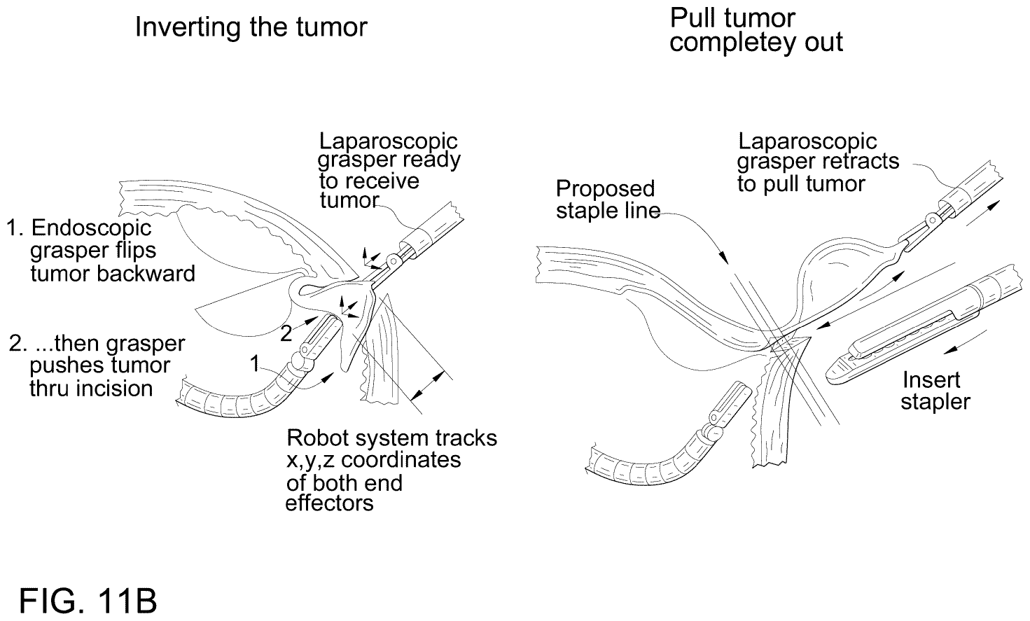

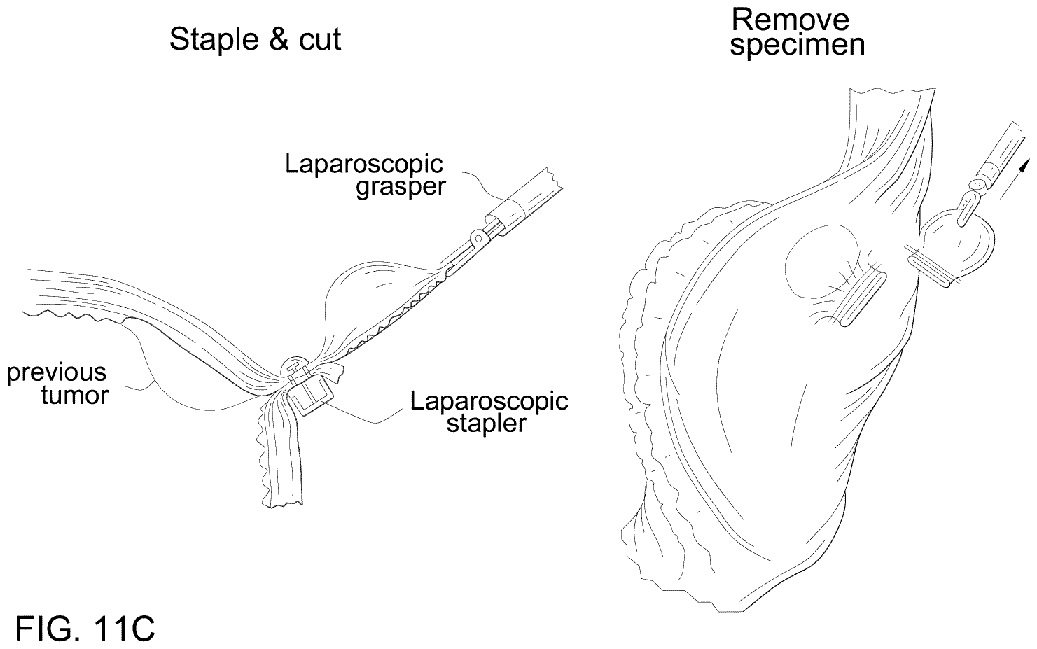

FIGS. 11A-C illustrate another example hybrid surgical procedure (e.g., a gastric tumor removal). As shown in FIG. 11A, an endoscopic grasper may be used to grasp a tumor in the stomach lining. As shown, the tumor may still be partially attached to the stomach. A laparoscopic incision device may be located on the outside of the stomach. The coordinates of the endoscopic grasper and the laparoscopic incision device may be known by an independent (e.g., robotic) system (e.g., a surgical hub). The laparoscopic incision device may be used to make an incision through the stomach.

As shown in FIG. 11B, once the incision has been made, the endoscopic grasper may be used to invert the tumor. The endoscopic grasper may push the tumor through the incision opening. A laparoscopic grasper may be used to grasp the tumor. The laparoscopic grasper may retract to pull the tumor through the incision opening. The independent system may track the coordinates of the endoscopic and laparoscopic graspers as they pass the tumor through the incision.

As shown in FIG. 11C, a surgical stapler (e.g., a laparoscopic stapler) may be used to staple the stomach lining to close the incision. The surgical stapler may cut the tumor from the stomach. The laparoscopic grasper may be retracted to remove the tumor from the patient's body.

One or more controllers may be used in combination to control one or more independent tools simultaneously. Example controllers may include one or more of: left/right hand controllers, left/right foot pedals, voice commands, and/or the like. For example, a left hand controller may operate an endoscopic device and a right hand controller may operate a laparoscopic device.

Dual simultaneous device motion may involve a device (e.g., one of the devices) being actively controlled (e.g., with assistance) by the system (e.g., semi- or fully-autonomously). For example, the controlled device may takes its lead of movement from another device (e.g., instrument) that is being actively controlled by a user.

Example actuation control schemes are provided herein. Actuation control may involve a force control (e.g., relative to a shared tissue contact). For example, the motion control parameter may be a force relative to a tissue contact shared between surgical instruments (e.g., a first and second surgical instrument).

A load control between two or more smart tissue control jaws (e.g., graspers) may be used. The load control may ensure an ideal amount of tissue tension is applied. The ideal amount of tissue tension may be an amount of tension that assists in endoscopic tissue dissection (e.g., sufficient tension to enable tissue separation without causing tissue tearing). Loads on the grasper shafts and/or jaws may be utilized for this function. The loads may be measured based on motor control feedback, by load sensors (e.g., integrated into the devices), and/or the like. The loads from the devices (e.g., individual devices) may be combined with the orientation of the devices. This combination may be used to estimate the amount of tension on the tissue between the devices.

Tissue tension can be automatically adjusted according to the endoscopic energy device feedback. Tension can be adjusted to optimize sealing or cutting. The Harmonic energy devices train users to not have tension on the organ/tissue/vessels when applying the energy. Utilizing a grasper or other instrument to be able to detect if load is being placed on the organ/tissue/vessel when applying energy could be used to alter the grasper or instrument to minimize the tension during the energy activation.

For example, a uterine manipulator may sense a force within the uterus as the uterus is displaced downward (e.g., as a laparoscopic instrument creates a tissue separation dissection of the outer wall of the uterus to the bladder). The force may be measured endoluminally on tissue that is being affected laparoscopically.

An external mechanism may measure the force on a main function element (e.g., the I-beam of an endocutter). The force measurement may be a motor source measured force (e.g., torque sensing of the output shaft or a proxy, for example, the current through the motor). The force measurement may be a load control on a trocar (e.g., holding a robot arm). The user may change the orientation of arms (e.g., relative to each other) by using the load control on the trocars (e.g., compared to on the instrument robot arms). That is, the trocar may be used as an extra joint for leverage or resisting the force applied by the instrument.

The motion control parameter may be associated with a positional relationship between surgical instruments (e.g., a first and second surgical instrument). A pre-defined positional relationship may be used for actuation control. For example, proximity control may be used for actuation control. The automated system may use feedback (e.g., from a camera) or other relational measurement between the actively controlled device and the automated control device to maintain a proximity of devices. The automated system may be capable of receiving adjustment commands (e.g., from a user) to modify the proximity.

For example, a laparoscopic sleeve gastrectomy may use tools together (e.g., to minimize unintended tissue trauma). During the procedure, the surgeon may utilize other tools (e.g., to be used in conjunction) to hold the tissue in a defined space (e.g., while an endocutter is navigated into position). The other tool(s) may move in a synchronized motion to not contact the endocutter. A grasper may hold the tissue up and away from other objects (e.g., to ensure that, as the device is fired, it does not pinch or staple unintended tissue to the staple line). If an endoscopic instruments (e.g., in the stomach, rectum, or bladder) approaches a lesion of interest; a robotic arm with a laparoscopic instrument (e.g., to be used for grasping and outer organ wall stability for the endoluminal resection or biopsy) may move with a set position of the endoscopic instrument.

Antagonistic positional station keeping control may be used for actuation control. An automated control arm may be capable of maintaining a three-dimensional positional location (e.g., station keeping). For example, the arm may be capable of maintaining the positional location even if forces are applied to the end-effector or the grasped tissue from an outside source. The position control may be capable of resisting externally applied force both in the direction of motion as well as opposite the direction of motion (e.g., without fluctuating).

For example, a powered articulation shaft may be designed to apply force in the direction of motion and resist forces applied opposite to the direction of motion. In a single link system, externally applied forces in the direction of motion may accelerate motion in that direction. An antagonistic system may have actuators in both directions (e.g., with a pre-defined force couple between the actuators). The differential of forces may drive motion. A force in either direction may add to the resisted load in that system opposed to that direction (e.g., the force may be reduced or slowed down if assisted, or increased or sped up if resisted).

Positional control of the control arm (e.g., not just the end-effector location) may be used for actuation control. A flexible endoscope may be able to hold a grasper in a known location. The endoscope may be able to hold the endoscope itself in a known location. Articulations may be used to position an organ in a desired location or orientation. The shaft of the device may provide a (pre) defined retraction or organ manipulation control (e.g., which may be as desired as the end-effector location or motions).

Hybrid load-strike control may be used for actuation control. Switchable state control may be used for actuation control. For example, load control may be used if displacements are small. If displacements over a (pre) defined amount (e.g., over a (pre) defined time), the device may operate in a position control mode.

A control loop with secondary limits may be used for actuation control. For example, position control with a maximum or minimum applicable force limit may be used for actuation control. Load control with a maximum displacement over a time and/or velocity limit may be used.

Dual loop control may be used for actuation control. For example, two separate/independent feedback monitors may be used. One may monitor the motor. One may monitor the driven part (e.g., the actual driven part) of the device.

In an example, the movable component may be a grasping device, and the motion event may be the second surgical instrument changing position, and a change in tissue tension associated with tissue held by the grasping device. In this case, the motion control parameter may be a tensile range, a displacement threshold, a window of time, and a range of distances between the first surgical instrument and the second surgical instrument. Adjusting motion of the movable component based on the motion control parameter and the motion event may involve, on a condition that the first surgical instrument moves a distance smaller than the displacement threshold during the window of time, adjust a load control of the movable component to keep the tissue tension within the tensile range. On a condition that the first surgical instrument moves a distance greater than the displacement threshold during the window of time, the device may move the movable component to keep a distance between the first surgical instrument and the second surgical instrument within the range of distances.

Control loop parameters may be used for actuation control. For example, proportionate, integral, derivative (PID) control loop parameters may be used. The proportionate parameter may be associated with a magnitude of force or velocity. The integral parameter may increase action in relation to the error and the time for which the error has persisted (e.g., duration of error). The derivative parameter may be associated with a magnitude of the error.

Predictive control may be used for actuation control. Predictive control may be based on one or more elements (e.g., three key elements). For example, predictive control may use a predictive model, an optimization in range of a temporal window, and/or feedback correction. The predictive model may be used to predict a future output based on historical information (e.g., about the process,) and/or an anticipated future input. For example, a state equation, transfer function, and/or a step or impulse response may be used as the predictive model.

In an example, an endoscopic grasping device may have a grip on a mucosal tumor (e.g., that has been mostly resected from inside the stomach). The endoscopic articulation may be used to control the tumor position and/or the orientation of the stomach (e.g., to prevent loss of acid control when a trans-wall incision is made). The user may move the receiving grasping device from the laparoscopic side into close proximity to the abdominal cavity side of the stomach wall. The receiving grasping device may take hold of the outside wall to control the stomach for the incision step. The laparoscopic grasper and the endoscopic grasper may be released for autonomous control. The endoscopic grasper may be given a proximity distance to maintain to the laparoscopic grasper. The laparoscopic grasper may be placed in a station keeping mode. For example, the device may receive an indication to enter a station-keeping mode. The device may maintain a global position of the moveable component in response to the indication.

A third laparoscopic blade (e.g., monopolar blade, conventional blade, or scissors) may be brought in to make a cut along the base of the still-intact connection of the tumor and the inside wall of the stomach. The blade force may be resisted by the laparoscopic grasper station keeping retraction. Orientation to prevent acid spill may be controlled by the endoscope (e.g., based on shape and the grasper holding the tumor base). Once the incision is made, the cutting element may be removed. The laparoscopic grasper may be used to grasp the tumor base from the laparoscopic side. The proximity control may be used to keep the endoscopic hold of the tumor relative to laparoscopic grasper (e.g., for fixation). The proximity distance may be adjusted (e.g., may be reduced to bring the devices closer together, if needed). Once the lap grasper has the base of the tumor, the endoscopic grasper may release its hold on the tumor. The endoscopic grasper may maintain (e.g., still hold) its position (e.g., thereby allowing the user-controlled laparoscopic grasper to flip the tumor to the abdomen space. The laparoscopic grasper may be placed in station keeping mode. The user may take control of the endocutter. The user may position the endocutter across the incision and the base of the tumor. The user may fire the endocutter. The user may then simultaneously cut the tumor loose and seal the incision. The endocutter may be placed in station keeping mode (e.g., while still clamped on the tissue). The endoscopic tools may be retracted (e.g., first). The laparoscopic grasper may be (e.g., may then be) placed in a user control mode. The endocutter jaws may be opened to enable removal of the tumor from the surgical site.

A laparoscopic endoscopic cooperative surgery (LECS) may be used for stomach tumor dissection. A tumor may be located adjacent to the esophageal sphincter on the greater curvature posterior side. Tumor removal may involve mobilization and retraction of the stomach into an irregular shape to access, dissect, and remove the tumor laparoscopically. Endoscopic sub-mucosal dissection with trans organ wall flexible endoscopic access may be combined with laparoscopic manipulation and specimen removal. Laparoscopic and endoscopic cooperative surgery may be used to remove gastric tumors.

A gastroscope (e.g., 5-12 mm overtube) may have working channel sizes of 2-4 mm and/or a local visualization scope. One or more (e.g., several) laparoscopic trocars, a laparoscope, and/or tissue manipulation and dissection instruments may be introduced (e.g., in an operating room).

Identification of the tumor location may be performed on the endoscope side. The endoscopic side may communicate the tumor location to the laparoscope side (e.g., where the stomach needs to be mobilized to allow for stomach retraction and manipulation). The gastroepiploic artery surrounds the perimeter of the stomach and is fixed to surrounding structures. The gastroepiploic artery may be freed to enable mobilization and separation of connective tissues. During this, bleeding may occur. The laparoscopic side may intervene if bleeding occurs.

The stomach may be manipulated and held in a position where the stomach acids are not over the portion of the stomach where the tumor resection will be performed (e.g., where the intra-cavity cut will be made). The stomach acids may be managed (e.g., with respect to gravity) to prevent inadvertent escape of the acids into the abdomen cavity.

Electro cautery may be used to free the perimeter of the mucosal layer within the stomach. Mucosal and sub-mucosal dissection may be expected (e.g., based on the depth of the tumor in the stomach wall). Inadvertent perforation of the serosal tissue layer may create leaks from the stomach to the abdomen. Controlled energy usage may be used to get deep enough to peel the tumor off (e.g., but not too deep to burn through the entire wall thickness).

Energy assisted dissection may be stopped with a portion of the tumor still connected to the stomach lining. The portion that is still connected may be pivoted through the incision (e.g., to keep hold of the tumor during extraction).

An incision may be made outside of the tumor margins (e.g., to allow for removal of the tumor from the laparoscopic side, and ensure that the cancer is removed). The location of the incision may be initiated from the laparoscopic side. The location of the incision may be coordinated with the endoscopic side and the tumor location. The tumor may be controlled and/or manipulated (e.g., during the incision creation) to prevent inadvertent cutting of the tumor. A tumor margin may be used to make sure that the retained tissue is cancer free. The tumor may be too large for oral extraction.

The tumor may be pivoted from the endoscopic space to the laparoscopic space. The stomach orientation may be controlled to prevent stomach acid from escaping into the abdomen. Localized bleeding may be controlled with advanced energy (e.g., from the laparoscopic and/or the endoscopic spaces). The tumor may be pivoted from the control and interaction of the endoscopic (e.g., endoluminal) instruments to the control and interaction of the laparoscopic instruments. During the hand-off there may be at least one point in time during which both sets of instruments are interacting with the same tumor tissue.

An endocutter may be introduced (e.g., from the laparoscopic side) and positioned to transect the tumor from the remaining stomach wall and seal the opening through which the tumor was passed. Poor positioning of the endocutter may result in a hole being made through the organ (e.g., that will need to closed before completion of the procedure). The stapler jaws may be overloaded by tissue thickness. In this case, the staples may be inadequately formed. The inadequately formed staples may not seal the organ (e.g., which may result in localized bleeding).

Limits may be added to create bounds for the assisting control. For example, in a transurethral resection of a bladder tumor (TURBT) (e.g., when the endorobotic instrument is near the tumor on the bladder wall), a laparoscopic device may be moved into position to affect a concave (e.g., from laparoscopic side) positioning of the tumor area (e.g., to assist in the transurethral resection). As another example, if a patient has been prepared for indocyanine green (ICG) fluoroscopy, the florescent may be used to monitor the proximity of the working robotic laparoscopic instrument to a surgical structure (e.g., a critical vessel, for example, during dissection of the liver lobe near the hepatic artery, dissection of the descending colon mesentery near the Inferior Mesenteric Artery (IMA), etc.)

A user may select a monitored parameter to operate the device movement within the closed loop control. A device (e.g., each device, for example, a grasper) may know which devices should move at a given time. For example, the device may be able to determine which device should move based on an operational window (e.g., which device has the space available, for example, based on tissue and/or organ proximity). The device in furthest proximity from a hazard or high risk area (e.g., carotid, nerves, critical structures, etc.) may be the device that should move. A predetermined hierarchy of control may be determined (e.g., prior to the procedure starting). The determination of which device should move may be based on a procedure plan and/or a user selection. The determination of which device should move may be based on a physical location of the device in proximity to a smart control system. For example, the grasper closest to controlling smart system may be the device that will move. In this case, the surgeon may know (e.g., always know) the grasper in closest proximity to the smart system. In some examples, a device on a dominant side/hand of the surgeon may be the device that is autonomously controlled.

When transecting a vessel using an energy device, graspers may work in unison with each other (e.g., and possibly a vision system) to control tissue tension. If too much tension is applied, hemostasis issues may occur. If too little tension is applied, undesired thermal damage may occur.

Force load control may be used if multiple (e.g., two) smart systems are holding the same piece of tissue. Position control may be used to help systems not bump into each other. A local command unit may regain command and/or delegate tasks to complete a procedural step.

To successfully hand off tissue from one device to another, the single control system may choreograph control (e.g., by calculating and displaying, to the user, an anticipated trajectory path for intercept, velocity, and distance to intercept). The control system may track (e.g., in real time) the movement of the devices compared to the anticipated/proposed device trajectory path. Defined paths may include the anticipation of next steps and/or obscured visibility (e.g., due to device, organ, or environmental interference).

If the trajectory path has an obstruction, the trajectory path may be updated. The updated trajectory path may be displayed to the user. The updated trajectory part may be a trajectory path (e.g., an ideal trajectory path) that bypasses the obstacle. If a visual condition is interrupted by an organ, which may be positioned with a smart retractor, the smart retractor may shift its position to maintain the desired visual condition.

If the user veers off the trajectory path, the system may reassess the trajectory path. The system may display the modified trajectory path (e.g., for hand off). For example, if the system cannot make a maneuver in the initial trajectory path, the system may recalculate the best direction(s) (e.g., updated trajectory path) and update trajectory instructions as needed. The control system may provide the user with a proposed or ideal trajectory path (e.g., if the currently anticipated path is an obstructed path or comes in close proximity to a critical structure). If the device(s) veer off the predetermined trajectory, the trajectory may be updated.

Predefined visual condition(s) may be identified. The visual conditions may be monitored (e.g., continuously monitored) and/or evaluated for interference. For example, during a gastric cancer procedure, the surgeon may want the stomach and/or the surrounding area visible and/or accessible (e.g., to dissect lymph nodes, remove the tumor, and/or reconstruct the stomach). In such a procedure, the liver may be lifted (e.g., with some form of organ retractor) to gain space and visibility. Smart organ retractors and/or smart graspers (e.g., utilized to retract organs) may be controlled based on a visual analysis of the surgical field. A visual condition may be interrupted by the organ itself shifting. The retractors may compensate for such an obstruction. The surgeon may shift the target tissue into a position that is no longer visible. If the liver shifts out of position, critical steps of the procedure may be interrupted. This may cause patient harm if the surgeon loses visibility.

The smart system displaying the trajectory path may display nearby structures (e.g., critical structures) to provide the user with awareness. If the trajectory path is in close proximity to a critical structure, the trajectory path may continue to be displayed along with a proposed trajectory path (e.g., to mitigate the potential hazard).

Cooperative alternating movements of a smart system may be used for anticipated events (e.g., next steps in a procedure). A common field of view may be maintained. The devices may rotate being stationary or changing position. A first system may hold its current position. A secondary system may move to the next anticipated area of visibility.

If a smart device approaches the outer extent of the vision system, the user may pause the device (e.g., momentarily). While the smart device is stationary, the vision system may reposition itself (e.g., to recenter the device within its frame). The system that will maintain its current position (e.g., and the system that will move) may be determined based on which vision system the current action is centered within. Using a combined registration, the vision system movement may be seamless. For example, the stationary system may be able to maintain fixed imaging on the current event, while the moving system may prepare for the next area of view (e.g., the direction of motion of the surgeon).

In an example, a surgeon may be separating an organ from surrounding tissue and may pause. The vision system may recognize the pause and anticipate that the surgeon will continue soon. The vision system may reduce its speed of movement (e.g., but continue to move) while the device is stationary (e.g., anticipating that the device will continue moving once the surgeon reengages). The vision system may anticipate the seam between the organ and tissue to follow (e.g., best fit follow) the surgeon's anticipated trajectory.

One or more camera systems (e.g., independent camera systems) may be affixed to the same laparoscope. The camera system may have independent rotation features to allow combined visualization within the same plane. Independent visualization systems or techniques may be used. For example, a visualization system may be a white light camera system, and/or an alternative imaging modality. For example, a procedure may use a C-arm and/or ultrasound that may relate back to a white light laparoscope. An endoscope may have multiple cameras (e.g., mounted within its tip). The cameras may track (e.g., independently track) devices that have been inserted and are moving within the body cavity.

A first and second vision system (e.g., vision system A and B) may be used. A critical event may occur that is centered in the vision system A. In this case, the vision system A may hold in one spot. The vision system B may move to include an area of visibility (e.g., a required area of visibility).

Those skilled in the art will appreciate that the features described herein may be implemented using any appropriate means for motion sensing/tracking. For example, motion sensing may be performed using sensing techniques based in ultrasonic, infrared, microwave, light detection and ranging (lidar), radio detection and ranging (radar), sound navigation and ranging (sonar), hybrid or dual-technology, and/or the like.

Some techniques for motion sensing may have benefits that are desirable in a surgical environment. For example, passive infrared sensors may be small, inexpensive, and use relatively low power. As another example, microwave sensors may be able to sense motion at farther distances than some other technologies.

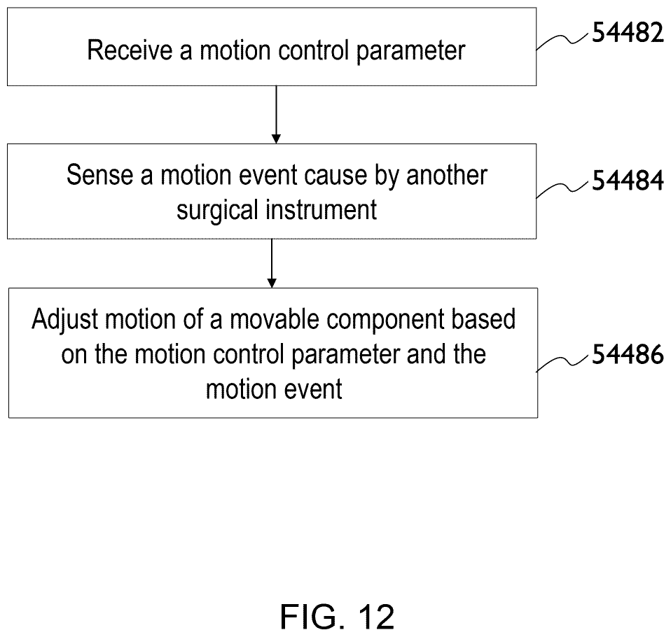

FIG. 12 illustrates an example method that may be performed by a surgical instrument. As shown, the method may involve, at 54482, receiving a motion control parameter. The motion control parameter may include one or more of: a force relative to a tissue contact shared between the first surgical instrument and the second surgical instrument; or a positional relationship between the first surgical instrument and the second surgical instrument. The method may involve, at 54484, sensing a motion event caused by a user control input to a second surgical instrument. The method may involve, at 54486, adjusting motion of the movable component based on the motion control parameter and the motion event.

What is claimed:

1. A first surgical instrument comprising a processor and a movable component, wherein the processor is configured to:
receive a motion control parameter, wherein the motion control parameter comprises one or more of: a force relative to a tissue contact shared between the first surgical instrument and the second surgical instrument; or a positional relationship between the first surgical instrument and the second surgical instrument;
sense a motion event caused by a user control input to a second surgical instrument; and
autonomously adjust motion of the movable component based on the motion control parameter and the motion event.

2. The first surgical instrument of claim 1, wherein the first surgical instrument is an endoscopic instrument, and wherein the second surgical instrument is a laparoscopic instrument.

3. The first surgical instrument of claim 1, wherein the processor being configured to autonomously adjust motion of the movable component based on the motion control parameter and the motion event comprises the processor being configured to:
determine an effect of the motion event based on a quantity defined by the motion control parameter; and
determine a way in which to adjust the motion of the moveable component to provide a corresponding canceling effect to cancel out the determine effect of the motion event.

4. The first surgical instrument of claim 1, wherein:
the movable component is a grasping device;
the motion event comprises a change in tissue tension associated with tissue held by the grasping device;
the motion control parameter comprises a tensile range; and
the processor being configured to adjust motion of the movable component based on the motion control parameter and the motion event comprises the processor being configured to adjust a load control of the movable component to keep the tissue tension within the tensile range.

5. The first surgical instrument of claim 1, wherein:
the motion event comprises the second surgical instrument moving away from the first surgical instrument;
the motion control parameter comprises a maximum distance between the first surgical instrument and the second surgical instrument; and
the processor being configured to adjust motion of the movable component based on the motion control parameter and the motion event comprises the processor being configured to move the movable component toward the second surgical instrument to keep a distance between the first surgical instrument and the second surgical instrument below the maximum distance.

6. The first surgical instrument of claim 1, wherein:
the motion event comprises the second surgical instrument moving towards the first surgical instrument;
the motion control parameter comprises a minimum distance between the first surgical instrument and the second surgical instrument; and
the processor being configured to adjust motion of the movable component based on the motion control parameter and the motion event comprises the processor being configured to move the movable component away from the second surgical instrument to keep a distance between the first surgical instrument and the second surgical instrument above the minimum distance.

7. The first surgical instrument of claim 1, wherein:
the movable component is a grasping device;
the motion event comprises the second surgical instrument changing position, and a change in tissue tension associated with tissue held by the grasping device;
the motion control parameter comprises a tensile range, a displacement threshold, a window of time, and a range of distances between the first surgical instrument and the second surgical instrument; and
the processor being configured to adjust motion of the movable component based on the motion control parameter and the motion event comprises the processor being configured to:
on a condition that the first surgical instrument moves a distance smaller than the displacement threshold during the window of time, adjust a load control of the movable component to keep the tissue tension within the tensile range; and
on a condition that the first surgical instrument moves a distance greater than the displacement threshold during the window of time, move the movable component to keep a distance between the first surgical instrument and the second surgical instrument within the range of distances.

8. The first surgical instrument of claim 1, wherein:
the moveable component comprises a scope;
the motion event comprises the second surgical instrument is moving out of a field of view of the scope;
the motion control parameter comprises a maximum distance that the second surgical instrument can be away from the center of the field of view of the scope; and
the processor being configured to adjust motion of the movable component based on the motion control parameter and the motion event comprises the processor being configured to move the scope to keep the second surgical instrument within the maximum distance away from the center of the field of view of the scope.

9. The first surgical instrument of claim 1, wherein the processor is further configured to:
receive an indication to enter a station-keeping mode; and
maintain a global position of the moveable component in response to the indication.

10. A method performed by a first surgical instrument comprising a movable component, wherein the method comprises:
receiving a motion control parameter, wherein the motion control parameter comprises one or more of: a force relative to a tissue contact shared between the first surgical instrument and the second surgical instrument; or a positional relationship between the first surgical instrument and the second surgical instrument;
sensing a motion event caused by a user control input to a second surgical instrument; and
autonomously adjusting motion of the movable component based on the motion control parameter and the motion event.

11. The method of claim 10, wherein the first surgical instrument is an endoscopic instrument, and wherein the second surgical instrument is a laparoscopic instrument.

12. The method of claim 10, wherein autonomously adjusting motion of the movable component based on the motion control parameter and the motion event comprises:
determining an effect of the motion event based on a quantity defined by the motion control parameter; and
determining a way in which to adjust the motion of the moveable component to provide a corresponding canceling effect to cancel out the determine effect of the motion event.

13. The method of claim 10, wherein:
the movable component is a grasping device;
the motion event comprises a change in tissue tension associated with tissue held by the grasping device;
the motion control parameter comprises a tensile range; and
adjusting motion of the movable component based on the motion control parameter and the motion event comprises adjusting a load control of the movable component to keep the tissue tension within the tensile range.

14. The method of claim 10, wherein:
the motion event comprises the second surgical instrument moving away from the first surgical instrument;
the motion control parameter comprises a maximum distance between the first surgical instrument and the second surgical instrument; and
adjusting motion of the movable component based on the motion control parameter and the motion event comprises moving the movable component toward the second surgical instrument to keep a distance between the first surgical instrument and the second surgical instrument below the maximum distance.

15. The method of claim 10, wherein:
the motion event comprises the second surgical instrument moving towards the first surgical instrument;
the motion control parameter comprises a minimum distance between the first surgical instrument and the second surgical instrument; and
adjusting motion of the movable component based on the motion control parameter and the motion event comprises moving the movable component away from the second surgical instrument to keep a distance between the first surgical instrument and the second surgical instrument above the minimum distance.

16. The method of claim 10, wherein:
the movable component is a grasping device;
the motion event comprises the second surgical instrument changing position, and a change in tissue tension associated with tissue held by the grasping device;
the motion control parameter comprises a tensile range, a displacement threshold, a window of time, and a range of distances between the first surgical instrument and the second surgical instrument; and
adjusting motion of the movable component based on the motion control parameter and the motion event comprises:
on a condition that the first surgical instrument moves a distance smaller than the displacement threshold during the window of time, adjusting a load control of the movable component to keep the tissue tension within the tensile range; and
on a condition that the first surgical instrument moves a distance greater than the displacement threshold during the window of time, moving the movable component to keep a distance between the first surgical instrument and the second surgical instrument within the range of distances.

17. The method of claim 10, wherein:
the moveable component comprises a scope;
the motion event comprises the second surgical instrument is moving out of a field of view of the scope;
the motion control parameter comprises a maximum distance that the second surgical instrument can be away from the center of the field of view of the scope; and
adjusting motion of the movable component based on the motion control parameter and the motion event comprises moving the scope to keep the second surgical instrument within the maximum distance away from the center of the field of view of the scope.

18. The method of claim 10, wherein the method further comprises:
receiving an indication to enter a station-keeping mode; and
maintaining a global position of the moveable component in response to the indication.
